(19) United States
(12) Patent Application Publication
(10) Pub. No.: US 2026/0060835 A1
(43) Pub. Date: Mar. 5, 2026

(54) PENILE TREATMENT DEVICE AND METHOD

(71) Applicant: Landon Trost, Orem, UT (US)

(72) Inventor: Landon Trost, Orem, UT (US)

(21) Appl. No.: 19/108,215

(22) PCT Filed: Sep. 12, 2023
(86) PCT No.: PCT/US2023/032493

§ 371 Date: Mar. 3, 2025

Related U.S. Application Data
(60) Provisional application No. 63/407,375, filed on Sep. 16, 2022.

(51) Int. Cl. A61F 5/41

(52) U.S. Cl. CPC A61F 5/41; A61F 2005/411; A61F 2005/412

(57) ABSTRACT

The present disclosure relates to a device and a method of applying traction and vacuum forces to a penis of a human body. The method includes coupling a traction device to a penis, applying a traction force to the penis using a traction assembly of the traction device, and applying a vacuum force to the penis.

### TECHNICAL FIELD

The present disclosure relates to methods and devices for penile traction, and in particular, methods and devices for penile traction assisted with a vacuum.

### BACKGROUND

Peyronie's disease is the development of fibrous scar tissue inside the penis that causes curved, painful erections. The most common signs and symptoms of Peyronie's disease include scar tissue that can be felt under the skin of the penis as flat lumps or a band of hard tissue, a significant bend or curvature to the penis, indents or pockmarks, a narrowing or an hourglass appearance, problems attaining or maintaining an erection, shortening of the penis, and pain, with or without an erection. Many of these symptoms affect one's ability to engage in sexual intercourse.

In severe cases, treatment for Peyronie's disease may be alleviated by traction exercises or stretching the penis. While traction may help correct curvature and lengthen the penis, traction does not affect girth. While penile pump devices are used to treat erectile dysfunction and increase blood flow and girth, penile pump devices are used on an as-needed basis, and can create new or exacerbate existing trauma in the penis. Other shortcomings with vacuum therapies include, for example, creating an hourglass shape by using a constriction band, and an inability to use the vacuum in combination with a drug therapy.

### SUMMARY

The present disclosure relates to devices and methods for treating penile aberrations related to Peyronie's disease and girth. In particular, the devices and methods described herein apply dynamic traction in combination with a vacuum to treat both penile aberrations and girth.

One general aspect includes a method of applying traction and vacuum forces to a penis of a human body. The method may include coupling a traction device to a penis and applying a traction force to the penis using a traction assembly of the traction device. The method may include applying a vacuum force to a housing, where the housing encloses the penis.

Another general aspect includes a device for applying traction and vacuum forces to a penis of a human body. The device includes a base that may include a first side configured for placement against the human body, and a second side opposite the first side. The device also may include a traction assembly configured to apply a traction force to a penis. The traction assembly may extend from the second side of the base and may be adjustable between a non-extended position to an extended position. The device may include a housing coupled to the base and defining an interior volume sized to enclose the traction assembly. The housing may be configured to define a vacuum chamber when a vacuum force is applied to the interior volume.

In another general aspect, a method of applying traction and vacuum forces to a penis of a human body may include applying a dynamic traction force to a penis, and applying a vacuum force to the penis.

In another general aspect, a system for applying traction and vacuum forces to a penis of a human body may include a traction assembly configured to apply a traction force to a penis. The traction assembly may be adjustable between a non-extended position and an extended position. A vacuum assembly may be coupleable to the traction assembly. The vacuum assembly may be configured for applying a vacuum force to a penis.

In one or more of the foregoing first, second, third, and fourth aspects, a device and method of applying traction and vacuum forces to a penis may include any one or more of the following aspects.

In one example, applying a vacuum force may include applying a vacuum force to an interior chamber of a housing, wherein the housing encloses the penis.

In another example, the method may include extending a linearly-adjustable portion of the traction assembly in a direction away from the human body.

In some examples, the method may include extending an expandable portion of the housing in the direction away from the human body.

In other examples, applying the vacuum force may include applying the vacuum force while the traction device is coupled to the penis and after applying the traction force.

In one form, applying the traction force may include applying a bending traction force to the penis.

In another form, applying the bending traction force may include rotating a bending portion of the traction assembly.

In some forms, applying the traction force may include applying a longitudinal traction force using a linearly-adjustable portion of the traction assembly.

In other forms, applying the bending traction force may include rotating a bending portion relative to the linearly-adjustable portion of the traction assembly.

In one example, the method may include rotating a distal housing portion relative to a proximal housing portion.

In another example, the linearly-adjustable portion may be received by the proximal housing portion.

In yet another example, the bending portion may be received by the distal housing portion.

In another example, rotating the bending portion may include pivoting the bending portion about a pivot axis.

In some examples, the pivot axis may be substantially perpendicular to a longitudinal axis of the bending portion.

In some examples, applying the bending traction force may include rotating a clamp of the traction assembly.

In other examples, the method may include coupling a housing to a base of the device before applying the vacuum force to the housing.

In one form, the method may include sealing a base of the traction device to the human body before applying the vacuum force.

In some examples, the base may be coupled to the traction assembly.

In another form, a seal may be disposed between the housing and the second side of the base.

In some forms, a pump may be coupled to the housing.

In another form, the pump may be configured to apply a vacuum to the interior chamber of the housing.

In other forms, the traction assembly may include a linearly-adjustable portion that is movable in a longitudinal direction.

In one example, the traction assembly may include a bending portion coupled to the linearly-adjustable portion.

In another example, the bending portion may be selectively rotatable relative to the linearly-adjustable portion.

In another example, the bending portion may be rotatable about a pivot axis.

In some examples, the pivot axis may be perpendicular relative to an axis of the linearly-adjustable portion.

In some examples, the bending portion may be adjustable between the first position, in which the bending portion is aligned with the linearly-adjustable portion, and the second position, in which the bending portion is angled relative to the linearly-adjustable portion.

In other examples, the housing may include a first part sized to receive the traction portion.

In some examples, the housing may include a second part sized to receive the bending portion.

In some forms, the first part may be coupled to the second part.

In one form, the second part of the housing may be selectively rotatable relative to the first part of the housing.

In another form, the second part of the housing may be removably coupled to the first part of the housing.

In some forms, one or more of the first part and the second part of the housing may be collapsible.

In other forms, the device may include a sealing element for sealing the base against the human body.

In one example, a cushion may be coupled to the first side of the base.

In some examples, the cushion may be configured for engaging with the human body.

In another example, the housing may be releasably coupled to the base.

### Definitions

As used herein, the term “about” means +/−10% of any recited value. As used herein, this term modifies any recited value, range of values, or endpoints of one or more ranges.

As used herein, the terms “top,” “bottom,” “upper,” “lower,” “above,” and “below” are used to provide a relative relationship between structures. The use of these terms does not indicate or require that a particular structure must be located at a particular location in the apparatus. Some examples may be described using the expression “coupled” and “connected” along with their derivatives. For example, some arrangements may be described using the term “coupled” to indicate that two or more elements are in direct physical or electrical contact. The term “coupled,” however, may also mean that two or more elements are not in direct contact with each other, but yet still co-operate or interact with each other. The examples described herein are not limited in this context.

Other features and advantages of the present disclosure will be apparent from the following detailed description, figures, and claims.

### BRIEF DESCRIPTION OF THE DRAWINGS

FIG. 1 is a front, perspective view of a penile traction and vacuum system or device in accordance with the teachings of the present disclosure, showing a traction assembly in a fully linearly-extended position, and a vacuum assembly;

FIG. 2 is a partial top view of the device of FIG. 1, showing the device in a non-extended position and partially illustrating the vacuum assembly;

FIG. 3 is a top view of the device of FIG. 2, showing the linearly-adjustable portion of the traction assembly in a partially, linearly-extended position;

FIG. 4 is a partial front, perspective view of the device of FIG. 1, showing a bending portion of the traction assembly in an extended position; and

FIG. 5 is a representative illustration of the device of FIG. 1 attached to a penis in a non-extended position.

### DETAILED DESCRIPTION

The present disclosure relates to systems, devices, and methods for treating penile aberrations related to Peyronie's disease. In particular, the systems, devices, and methods described herein apply traction therapy in combination with vacuum therapy to treat both penile aberrations and girth. Traction therapies disclosed herein include applying longitudinal and/or contralateral penile traction forces to treat decreased penile length and penile curvature. In particular, the traction forces applied are dynamic loads such that as the penis stretches in response to the traction forces, additional traction forces continue to be applied. Applying vacuum with traction may reduce time needed for traction exercises and can treat penile girth.

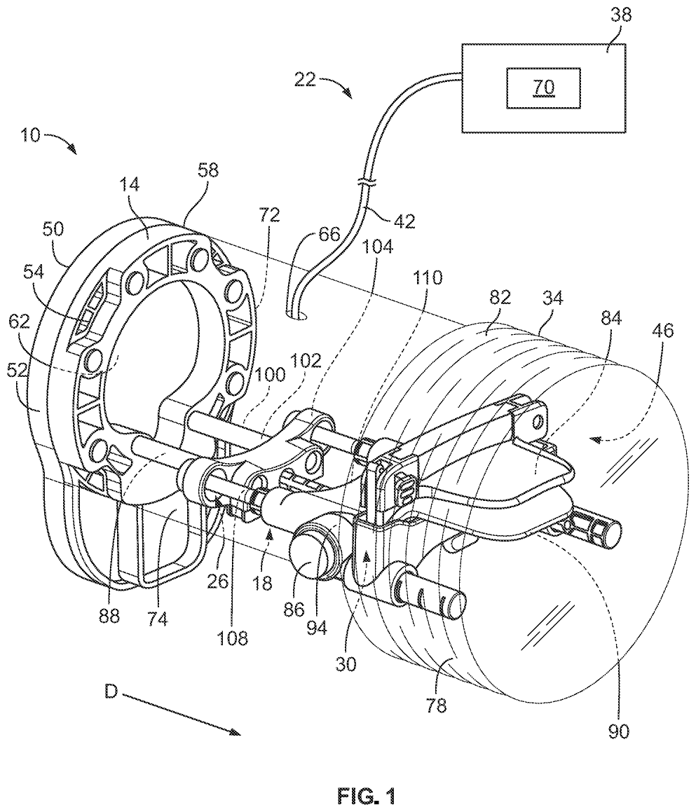

In FIG. 1, an example system or device 10 for applying penile traction and vacuum forces can be used to provide penile length modification, girth modification, and/or to treat penile aberrations. Such aberrations may be related to, but are not necessarily exclusively related to, Peyronie's disease. In some implementations, the aberrations of the penis may be, but are not limited to, decreased penile length, decreased penile girth, hour-glass shape, detents, and/or excessive penile curvature. In some examples described herein, the penile traction and vacuum system 10 is configurable to treat one or more of decreased penile length, excessive penile curvature, hour-glass shape, detents, and girth. The system 10 is configurable for sale and/or packaging together including a vacuum assembly and traction assembly, and is therefore also referred herein as a “penile traction and vacuum device” or simply “device.” However, in some examples, a vacuum assembly of the illustrated system may be used with an existing traction assembly or device. Thus, in some examples, the “device” may refer to a system of separate assemblies working together to apply traction and vacuum forces to a penis. The examples described herein are not limited in this context.

The penile traction and vacuum device 10 in FIG. 1 includes a base 14, a traction assembly 18, and a vacuum assembly 22. The traction assembly 18 extends away from the base 14 and is movable between a non-extended position and various extended positions. To allow customization, the traction assembly 18 includes a linearly-adjustable portion 26 and a bending portion 30 coupled to the linearly-adjustable portion 26. Each of the linearly-adjustable portion 26 and the bending portion 30 may be non-extended and extended. The vacuum assembly 22 includes a housing 34 and a pump 38 coupled to the housing 34 by a line 42. The housing 34 is coupled to the base 14 and defines an interior chamber 46 sized to enclose the penis and, either partially or entirely, the traction assembly 18. The interior chamber 46 of the housing 34 becomes a vacuum chamber when operating the pump 38. The device 10 is configurable so that one or more of the linearly-adjustable portion 26 and the bending portion 30 may be utilized together with the vacuum assembly 22. The vacuum assembly 22 can be retro-fitted to an existing traction device, or may be integrally formed with the base 14 and/or the traction assembly 18.

The base 14 has a cushioned, first side 50 that is configured for comfortable placement against a human body, and a second side 54 opposite the first side 50. When the penile traction device 10 is worn by a user, the cushion 52 creates a seal with a skin surface of the user. An open, proximal end 58 of the housing 34 is sealed to the second side 54 of the base 14 and extends in a distal direction D. The base 14 defines an opening 62 through which the user's penis can pass, and the proximal end 58 of the housing 34 is disposed around the opening 62. In the illustrated example, the base 14 has a generally circular ring shape and a key-shaped opening 62. However in other examples, the base 14 and/or opening 62 can have a U-shape, V-shape, ovular shape, and the like. The base 14 may be pre-assembled with the traction assembly 18, or may be pre-assembled with the housing 34 of the vacuum assembly 22. In this context, “proximal” means towards the user, while “distal” means away from the user.

The pump 38 of the vacuum assembly 22 is in fluid communication with the interior chamber 46 of the housing 34 at a port 66. The pump 38 includes a user interface 70 or display, such as, for example, a touch-screen or a readout dial, so that a user can operate, control, and/or monitor a vacuum pressure applied to the interior chamber 46 of the device 10. In some examples, the pump 38 may be communicatively coupled (e.g., wireless connection) with, and operated by, a remote device such as, for example, a user's mobile device. In the illustrated example, the port 66 is disposed on a top side of the housing 34. However, in other examples, the port 66 may be disposed on a bottom side, or lateral sides of the housing 34. Further, while the pump 38 is externally disposed relative to the housing 34, in other examples, the pump 38 may be built into the device 10 itself (i.e., in a portion of the housing 34 or in the base 14).

The housing 34 of the vacuum assembly 22 is removably coupled to the base 14 to facilitate assembly, operation, and disassembly of the traction assembly 18. The base 14 may include a groove or other mating connection to receive the proximal end 58 of the housing 34. For example, the proximal end 58 of the housing 34 couples to the base 14 by interference fit, snap-fit, adhesive, hook and loop, or by another coupling mechanism arranged in or with the base 14. To ensure an airtight seal with the base 14, the housing 34 of the vacuum assembly 22 of the device 10 sealably couples to the base 14 to create an air-tight interior chamber 46 within the housing 34. Specifically, a seal 72 is disposed between the housing 34 and the base 14, and securably engages the housing 34 to the base 14.

The housing 34 is sized to enclose the traction assembly 18 and maintain a fluid tight vacuum chamber while the traction assembly 18 translates and/or rotates. The housing 34 includes a first part 74 coupled to the base 14 and sized to receive the linearly-adjustable portion 26 of the traction assembly 18. A second part 78 of the housing 34 is coupled to the first part 74 at a seam 82 and is sized to receive the bending portion 30 of the traction assembly 18. Specifically, the first and second parts 74, 78 of the housing 34 can flexibly expand, collapse, twist, and/or bend independently from each other to accommodate the various movements and positions of the traction assembly 18. The housing 34 is formed from a transparent, flexible and collapsible material. The first part 74 may be removably coupled to the second part 78 at the seam 82 to facilitate assembly of the device 10, or the first and second parts may be a singular housing connected by a flexible joint (i.e., seam 82). In the illustrated example, the housing 34 is collapsible and expandable much like a vacuum hose. In other examples, the housing 34 may have telescoping parts to accommodate linear and rotational extensions. In the telescoping example, the housing 34 may include one or more seals.

The second part 78 of the housing 34 has a wider dimension than the first part 74 to accommodate a clamp 84 (described further below) of the bending portion 30. The first and second parts 74, 78 are substantially cylindrical, however, in other examples, the first and second parts 74, 78 of the housing 34 may be frustoconical, ellipsoidal, or of another shape. In other non-limiting examples, the first and second parts 74, 78 of the housing 34 may be similarly dimensioned and constructed, may be sized to fit entirely over the traction assembly 18, the base 14, or both. In such an example, the housing 34 would be constructed to sealingly engage with the body of the patient.

As shown in FIGS. 1-3, the first part 74 of the housing 34 distally extends from the base 14 and over a proximal end 88 of the traction assembly 18 to the seam 82, which is disposed adjacent to first and second release buttons 86 (described further below) of the traction assembly 18. The second part 78 of the housing 34 extends in the distal direction D from the release buttons 86 and over a distal end 90 of the traction assembly 18. The seam 82 connects the first and second parts 74, 78 of the housing 34 and seals with a button housing 94 of the release buttons 86. The seam 82 of the housing 34 provides access to the release buttons 86 of the traction assembly 18 without compromising the integrity of the vacuum-sealed interior chamber 46. In other non-limiting examples, the seam 82 may be arranged to cover the buttons 86.

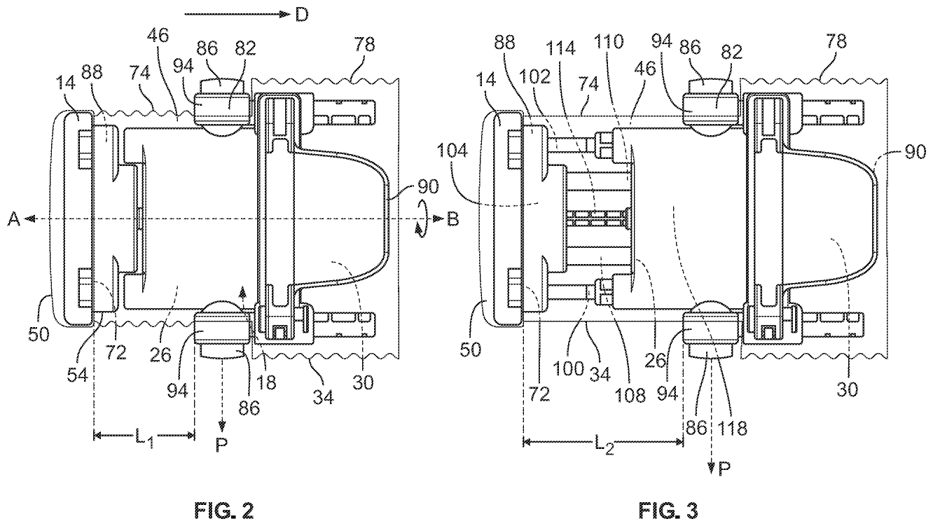

Referring to FIGS. 2 and 3, the device 10 is respectively shown in an initial, non-extended position and a second, linearly-extended position. In the non-extended position of FIG. 2, the first part 74 of the housing 34 is in a collapsed configuration having a collapsed length L1 and the linearly-adjustable portion 26 of the traction assembly 18 is in a non-extended position. In FIG. 3, the first part 74 of the housing 34 is in an extended position, expanding to a length L2 to accommodate the extension of the linearly-adjustable portion 26. In both FIGS. 2 and 3, the second part 78 of the housing 34 remains in a collapsed position because the bending portion 30 of the traction assembly 18 is in a non-extended position.

The traction assembly 18 may be operated while disposed within the housing 34, or may be operated, and then later coupled to the vacuum assembly 22. When the linearly-adjustable portion 26 is aligned with the bending portion 30, a longitudinal axis A of the linearly-adjustable portion 26 is substantially parallel with a longitudinal axis B of the bending portion 30, as shown in FIG. 2. The bending portion 30 can be adjusted relative to the linearly-adjustable portion 26 for comfort by angling the bending portion 30 relative to the linearly-adjustable portion 26. In some cases, an angle between the axes may be, without limitation, in a range of about 0° to about 5°, or about 1° to about 4°, or about 2° to about 3°, or about 0° to about 10°. As will be described further below, an angle between the linearly-adjustable portion 26 and the bending portion 30 can increase for correcting penile curvature.

The linearly-adjustable portion 26 extends distally from the base 14 by a user-adjustable distance. A portion of linearly-adjustable portion 26 is spring-loaded along the longitudinal axis A of the linearly-adjustable portion 26 such that linearly-adjustable portion 26 can apply a dynamic longitudinal traction force to the user's penis. The linearly-adjustable portion 26 includes two stages of longitudinal length adjustability. A first stage of linear length adjustability extends distally from the base 14, and a second stage of linear length adjustability extends distally from first stage in a telescoping manner.

Turning back to FIG. 1, the first stage of longitudinal length adjustability includes a first and second extension rods 100, 102 and a yoke 104. The first and second extension rods 100, 102 extend distally from the base 14, and the yoke 104 is slidably coupled to the first and second extension rods 100, 102. The yoke 104 is configured to releasably lock in a plurality of longitudinal positions along the lengths extension rods 100, 102. In some examples, the base 14 is configured with multiple mounting holes to accommodate the mounting of first and second extension rods 100, 102 at various locations on base 14. The first and second extension rods 100, 102 of various lengths and with various geometries can be used to accommodate various penis sizes.

The second stage of longitudinal length adjustability includes three rods 108, 110, 114 coupled to, and extending distally from, the yoke 104, as shown in FIG. 3. Third and fourth extension rods 108, 110 slidably terminate within a tensioner sub assembly 118. Two compression springs are within the tensioner sub assembly 118 (hidden from view), and are slidably coupled with the extension rods 108, 110. The compression springs tend to bias tensioner sub assembly 118 distally away from yoke 104 to apply a dynamic tensile force (i.e., a longitudinal traction force that adjusts along with adjustments in length of the linearly-adjustable portion 26) to a penis while a user is wearing penile traction device 10.

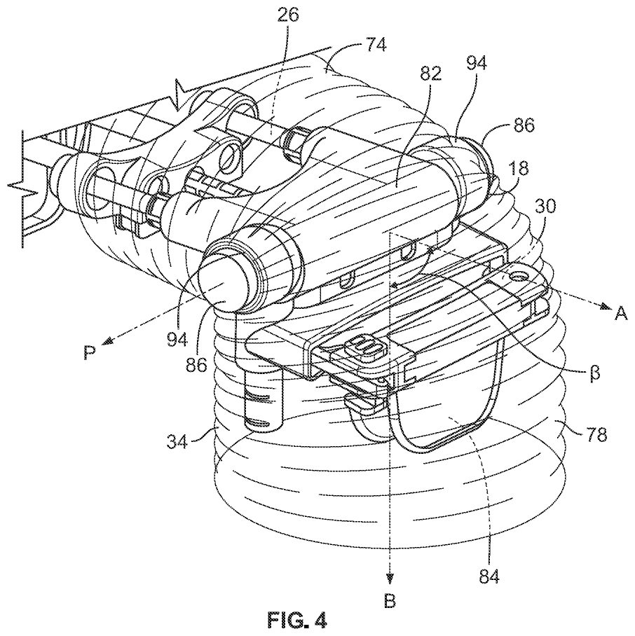

The bending portion 30 is selectively rotatable relative to the linearly-adjustable portion 26 and the base 14 in two ways. First, the bending portion 30 may be rotated to adjust orientation of the bending portion 30 relative to the linearly-adjustable portion 26 about the longitudinal axis B, as shown in FIG. 3. For example, the bending portion 30 may rotate about the longitudinal axis B based on a desired orientation to effectively treat a particular penile curvature. In this first way, the bending portion 30 is configured to rotate relative to, while maintaining alignment with, the linearly-adjustable portion 26. Second, and as shown in FIG. 4, the bending portion 30 may be pivoted relative to the linearly-adjustable portion 26 about a pivot axis P such that the bending portion 30 is angled (i.e., angle β) relative to the linearly-adjustable portion 26. The second part 78 of the housing 34 also bends relative to the first part 74 to accommodate the various incremental angled positions of the bending portion 30.

In FIG. 4, the bending portion 30 pivots relative to the pivot axis P that is substantially perpendicular to the bending portion axis B and the linearly-adjustable portion axis A. The bending portion 30 includes the clamp 84, which is configured to releasably couple a distal portion of the user's penis to the traction assembly 18. To pivot the bending portion 30 relative to linearly-adjustable portion 26, the user can engage the buttons 86 and freely pivot the bending portion 30 about the P axis. As the bending portion 30 is pivoted relative to the linearly-adjustable portion 26, the A axis of linearly-adjustable portion 26 becomes nonparallel relative to the B axis of the bending portion 30. An angle β is defined between the A and B axes. In some examples, the bending portion 30 can be releasably latchable so that the angle β can be adjusted at about 15° increments between 0° and 120°; at about 20°, at about 30°, at about 45°, or at about 60° increments between 0° and 120° (or greater than 120° in some examples). The device 10 may be configured to provide a dynamic bending traction force by pivoting the bending portion 30 relative to the linearly-adjustable portion 26.

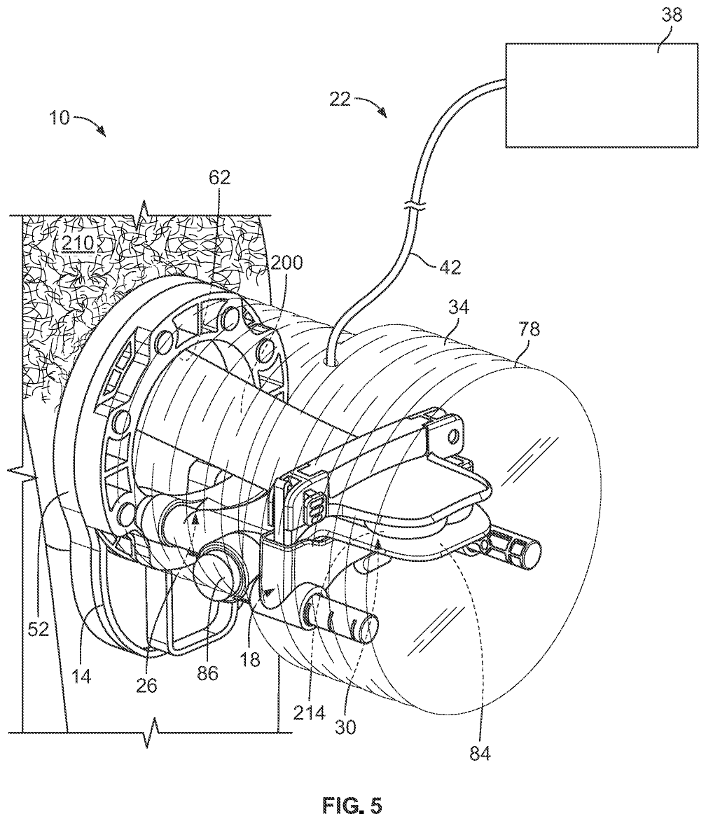

The present disclosure encompasses methods of using the device 10 of FIGS. 1-5 and a method of applying traction and vacuum forces to a penis. A method of treating a penis using the traction and vacuum device 10 initially includes configuring and assembling the device 10 to meet the needs and comfort of the patient. This step includes choosing particular sized rods and attaching them to the base 14 in one of a number of different manners and/or rotating the bending portion 30 relative to the linearly-adjustable portion 26, to treat penile curvature, if necessary. Once the device 10 is customized to the patient's needs, in FIG. 5 the method includes inserting a penis 200 of a patient 210 through the opening 62 in the base 14 and engaging the cushion 52 of the base 14 with the lower abdomen of the patient 210 to create a seal. A distal tip 214 of the patient's penis 200 is then secured to the clamp 82 of the traction assembly 18. At this point, linear traction forces and/or rotational traction forces may be applied to the user's penis 200 by moving the traction assembly 18 between various non-extended and extended positions. This may include, for example, adjusting the respective portions of the traction assembly 18 to extend longitudinally and rotationally.

The vacuum housing 34 may be applied after the penis 2 is stretched by disposing the first part 74 of the housing 34 over the linearly-adjustable portion 26 of the traction assembly 18 and disposing the second part 78 of the housing 34 over the bending portion 30 of the traction assembly 18. The seam 92 is configured to sealingly engage with the button housing 94 of the traction assembly 18. After the housing 34 sealingly engages with the base 14, the patient may then apply a vacuum force to the penis by connecting the pump 38 to the housing 34 and then operating the pump 38 according to a treatment regimen. A method of applying both traction and vacuum forces may be performed in other manners with different devices and tools.

In some examples, various components of penile traction device 10 are readily interchangeable with replacement components that can differ in size, material properties, shape, and the like. In some examples, penile traction device 10 can be assembled in two or more manners, or can be otherwise adjustable, so as to facilitate customization for various users.

The device 10 of FIGS. 1-5 may include features or components related to the adjusting the rotational and linear components of the traction assembly disclosed in U.S. Pat. Nos. 10,881,545 and 10,117,771, the entire contents of which are incorporated herein by reference. Accordingly, one or more design features described in the context of the penile traction devices disclosed in U.S. Pat. Nos. 10,881,545 and/or 10,117,771 can be alternatively or additionally included in the penile traction device examples described herein. Features of the penile traction devices disclosed in U.S. Pat. Nos. 10,117,771 and 10,881,545, that are directed to aspects such as, but not limited to, adjustability, modularity, force measurement, tensioning, contralateral traction, clamping, and the like, can be alternatively or additionally included in the penile traction and vacuum device examples described herein. Any and all such hybrid designs and combinations of features are within the scope of this disclosure.

In some examples, the base 14 and/or the housing 34 are made of one or more polymeric materials, such as, for example, DELRIN®, polystyrene, acrylonitrile butadiene styrene, polyvinyl chloride, polyethylene, high density polyethylene, low density polyethylene, polypropylene, polycarbonate, polyphenelyne ether, polyamide (PA or Nylon), ultra high molecular weight polyethylene, polyimide, polyetherimide, polyphenylene sulfide, polyurethane, polyetheretherketone, thermoplastic copolyether (PEBAX), Fluorinated Ethylene Propylene, and combinations thereof. Alternatively, the base 14 can be made of metals such as aluminum, stainless steel, titanium, and the like, and alloys thereof. Further, in some examples coated metals are used for base 14, such as, for example, silicon-coated aluminum. It should be understood that the forgoing materials are just some example materials that can be used to make base 14 and/or housing 34, and that other materials (e.g., graphite, natural materials, etc.) are also within the envisioned scope of this disclosure. Moreover, the traction assembly 18, without limitation, can be constructed of any one or more of the materials listed above.

The disclosed penile traction and vacuum device 10 and method disclosed herein advantageously treat multiple penile aberrations related to various underlying causes. The device 10 and method may be customizable, by either activating certain features or adjusting the device to fit a user comfortably, so that the device 10 and method can treat a variety of patients and their particular needs. For example, the traction assembly 18 may be customized to focus on either lengthening or curvature, and may include either the linearly-adjustable portion or the bending portion. The vacuum assembly 22 may be attached or detached based on the need of the patient and the vacuum housing 34 is designed to accommodate the linearly-adjustable portion 26 and/or the bending portion 30. Further, the vacuum assembly 22 described herein is configurable to retrofit onto an existing traction device.

The device 10 and method disclosed herein combine traction and vacuum forces to treat patients with Peyronie's disease that would also like to increase girth. Current treatments for Peyronie's disease do not affect penile girth. In fact, patients with Peyronie's disease are typically discouraged from using penile pumps because the existing technology can create more trauma to the penis (e.g., create an hour-glass shape) and even exacerbate the aberrations resulting from Peyronie's disease. The presently disclosed device 10 and method avoid creating new trauma and/or worsening existing conditions by applying a vacuum force to the penis in a less traumatizing manner. Further, the device 10 and method of the present disclosure may be used in combination with drug therapies to treat Peyronie's disease.

In some further circumstances, the device 10 and method provided herein are beneficial for use pre-operatively or during post-operative recovery following surgical treatment of Peyronie's disease or other urological conditions such as prostate cancer (e.g., use for post-prostatectomy erectile function rehabilitation). In general, the treatment algorithm for Peyronie's disease can include the use of a penile traction and vacuum device. The goal of such therapy is to increase penile length or at a minimum restore length that was previously lost to the disease state and increasing girth. Traction and vacuum therapies can be used in one or more phases of treatment including prior to or following surgery, during or after injection treatments, or as a stand-alone therapy. The device 10 and method described herein may also help improve underlying erectile function and/or other conditions.

There are also other indications for the use of penile traction and vacuum therapy. One such use is for reducing penile curvature, either as a standalone therapy or in combination with injections and/or surgery. Another indication for the penile traction devices provided herein would be for men scheduled to undergo placement of a penile prosthesis. Use of a penile traction device may increase the overall length of the patient's anatomy possible allowing for a larger prosthesis to be inserted, and also potentially make the surgery itself easier. A third indication is to use the traction devices as a stand-alone therapy. Many men complain of decreased penile length, particularly among those experiencing erectile dysfunction from any one of several known contributing conditions (diabetes, following prostatectomy, vascular disease, and others), and this therapy has been shown to increase length without need for other procedures.

While this specification contains many specific implementation details, these should not be construed as limitations on the scope of any invention or of what may be claimed, but rather as descriptions of features that may be specific to particular examples of particular inventions. Certain features that are described in this specification in the context of separate examples can also be implemented in combination in a single example. Conversely, various features that are described in the context of a single example can also be implemented in multiple examples separately or in any suitable subcombination. Moreover, although features may be described herein as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous.

Moreover, the separation of various system modules and components in the examples described herein should not be understood as requiring such separation in all examples, and it should be understood that the described program components and systems can generally be integrated together in a single product or packaged into multiple products.

Particular examples of the subject matter have been described. Other examples are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain implementations, multitasking and parallel processing may be advantageous.

1-30. (canceled)

31. A method of applying traction and vacuum forces to a penis of a subject, the method comprising:
applying a bending traction force to the penis using a traction assembly of a traction device coupled to the penis; and
applying a vacuum force to the penis.

32. The method of claim 31, wherein applying the vacuum force comprises applying the vacuum force to an interior chamber of a housing of the traction device, wherein the housing encloses the penis.

33. The method of claim 32, comprising extending a linearly-adjustable portion of the traction assembly in a direction away from the subject.

34. The method of claim 33, comprising extending an expandable portion of the housing in the direction away from the subject.

35. The method of claim 31, wherein applying the vacuum force comprises applying the vacuum force while the traction device is coupled to the penis and after applying the bending traction force.

36. The method of claim 31, wherein applying the bending traction force comprises rotating a bending portion of the traction assembly.

37. The method of claim 31, comprising applying a longitudinal traction force using a linearly-adjustable portion of the traction assembly.

38. The method of claim 37, wherein applying the bending traction force comprises rotating a bending portion relative to the linearly-adjustable portion of the traction assembly.

39. The method of claim 38, comprising rotating a distal housing portion relative to a proximal housing portion of the traction device, wherein the linearly-adjustable portion is received by the proximal housing portion and the bending portion received by the distal housing portion.

40. The method of claim 31, wherein applying the bending traction force comprises rotating a clamp of the traction assembly.

41. A device for applying traction and vacuum forces to a penis of a subject, the device comprising:
a base comprising a first side configured for placement against the subject, and a second side opposite the first side;
a traction assembly configured to apply a traction force to the penis of the subject, the traction assembly comprising a bending portion and extending from the second side of the base and being adjustable between a first position and a second position; and
a housing coupled to the base and defining an interior chamber sized to receive the traction assembly,
wherein the housing is configured to define a vacuum chamber when a vacuum force is applied to the interior chamber.

42. The device of claim 41, comprising a pump coupled to the housing and configured to apply a vacuum to the interior chamber of the housing.

43. The device of claim 41, wherein the traction assembly comprises a linearly-adjustable portion that is movable in a longitudinal direction.

44. The device of claim 43, wherein the bending portion is coupled to the linearly-adjustable portion and is selectively rotatable relative to the linearly-adjustable portion.

45. The device of claim 44, wherein the bending portion is rotatable about a pivot axis, the pivot axis perpendicular relative to an axis of the linearly-adjustable portion.

46. The device of claim 44, wherein the bending portion is adjustable between the first position, in which the bending portion is aligned with the linearly-adjustable portion, and the second position, in which the bending portion is angled relative to the linearly-adjustable portion.

47. The device of claim 44, wherein the housing comprises a first part sized to receive the linearly-adjustable portion and a second part sized to receive the bending portion, the first part coupled to the second part.

48. The device of claim 47, wherein the second part of the housing is selectively rotatable relative to the first part of the housing.

49. The device of claim 47, wherein one or more of the first part and the second part of the housing is collapsible.

50. The device of claim 41, wherein the housing is releasably coupled to the base.
